(19) United States
(12) Patent Application Publication
(10) Pub. No.: US 2022/0204344 A1
(43) Pub. Date: Jun. 30, 2022

(54) PROCESS FOR PREPARING CUBIC PI-PHASE MONOCHALCOGENIDES

(71) Applicant: B.G. NEGEV TECHNOLOGIES AND APPLICATIONS LTD., AT BEN-GURION UNIVERSITY, Beer-Sheva (IL)

(72) Inventors: Yuval GOLAN, Omer (IL); Ran Eitan ABUTBUL, Lehavim (IL); Elad SEGEV, Petach Tikva (IL); Uri ARGAMAN, Sderot (IL); Guy MAKOV, Omer (IL); Eyal TOUTIAN, Tel Aviv (IL)

(21) Appl. No.: 17/601,465

(22) PCT Filed: Apr. 16, 2020
(86) PCT No.: PCT/IL2020/050440

§ 371 Date: Oct. 5, 2021

Related U.S. Application Data
(60) Provisional application No. 62/886,371, filed on Aug. 14, 2019.
(60) Provisional application No. 62/834,413, filed on Apr. 16, 2019.

(51) Int. Cl. C01B 19/00; C07C 211/03; C01G 19/00

(52) U.S. Cl. CPC C01B 19/007; C07C 211/03; C01G 19/00; C01P 2004/03; C01P 2002/72; C01P 2004/04; C01P 2002/76

(57) ABSTRACT

The invention provides process for preparing tin or germanium monochalcogenides of cubic crystalline structure, the process comprises combining a source of tin or germanium and a source of chalcogenide in a reaction vessel in the presence of uncharged liquid primary amine R—NH₂ and a charged form R—NH₃+ associated with a counter anion, wherein R is saturated or unsaturated hydrocarbyl, which may be the same or different in the uncharged and charged forms, and recovering from the reaction mixture an essentially pure cubic phase of the monochalcogenides.

Compounds composed of elements of groups 14 and 16 of the periodic table (named monochalcogenides; i.e., of the formula MX, where M for example is Sn or Ge, and X is S or Se), are an attractive class of semiconductors that is being considered in recent years for their potential incorporation in various applications such as solar cells, near-infrared (NIR) detectors, and optoelectronic devices.

The abovementioned monochalcogenides were known to exist in orthorhombic crystal structure (the α-phase). However, it has been recently shown that these monochalcogenides have cubic polymorphs (designated π-phase). The first member of the cubic polymorphs to be discovered was π-SnS, which was synthesized in the form of nanoparticles and described by our research group in the year 2015 [A. Rabkin, S. Samuha, R. E. Abutbul, V. Ezersky, L. Meshi and Y. Golan, Nano Lett., 2015, 15, 2174-2179]. We reported on the exceptionally large cubic unit-cell and atomic positions of a newly discovered SnS phase which was designated as the n phase. The new cubic polymorph is significantly different from the conventional α-SnS, that is, of the orthorhombic crystal structure.

Subsequently, it was shown that the n phase is not limited only to the Sn—S material system. Experimental studies carried out in the SnSe system has shown that π-SnSe can also be obtained either in the form of nanoparticles [R. E. Abutbul, E. Segev, S. Samuha, L. Zeiri, V. Ezersky, G. Makov and Y. Golan, CrystEngComm, 2016, 18, 1918-1923] or thin films [P. K. Nair, E. Barrios-Salgado and M. T. S. Nair, Phys. Status Solidi A, 2016, 213, 2229-2236; R. E. Abutbul, A. R. Garcia-Angelmo, Z. Burshtein, M. T. S. Nair, P. K. Nair, Y. Golan, “The Puzzle Unraveled: Crystal Structure of Cubic Tin Sulfide in Thin Films”, CrystEngComm 18 (2016) 5188-5194].

Our previously reported synthesis of nanoparticles of cubic tin monochalcogenides is based on the ‘hot injection’ method, which consists of preparing separate hot solutions of stannous salt (e.g., SnCl₂) and a source of the chalcogen, each dissolved in oleylamine, followed by injection of the latter solution to the former. That is, the primary amine (oleylamine) acts as the reaction solvent in addition to its role as a surface active substance (surfactant). The reaction is marked by instantaneous colour change, e.g., from transparent yellowish to opaque deep-brown solution. This approach resulted in co-existence of both cubic (n) and orthorhombic (a) phases in the reaction product at varying proportions.

The major goal of the present invention is to improve on the abovementioned synthetic pathway and provide a manageable process, amenable to large scale production, leading invariably to the formation of essentially polymorphically pure cubic monochalcogenides (e.g., cubic tine sulphide or tin selenide). By “essentially polymorphically pure cubic monochalcogenides” is meant a reaction product (for example, in the form of nanoparticles) consisting of not less than 70%, and preferably not less than 75%, e.g., not less than 85%, not less than 95% and more preferably not less 99% of the cubic crystalline π-phase. Most preferably, the reaction product is free of the α-phase.

We have now found that the monochalcogenide formation reaction, which takes place in primary amine solvent, is strongly influenced by the presence of moisture and carbon dioxide contaminates. For example, unintentional traces of water in the reaction could come from hydrated reaction solvent, hydrated metal salts, and glassware not thoroughly dried. Water causes the release of HCl to the reaction medium through reaction with the tin source, SnCl₂. In turn, HCl reacts with the amine reaction solvent, oleylamine, transforming it from neutral to charged surfactant, namely, the corresponding ammonium chloride salt. The overall reaction is represented by the following equation (R—NH₂ indicates the primary amine reaction solvent):

6SnCl₂+8H₂O+12R—NH₂→Sn₆O₄(OH)₄+12R—NH₃⁺Cl⁻

A similar path occurs in the case of CO₂, which reacts with primary amine (such as oleylamine), to give charged molecular pairs shown by the following reaction equation:

CO₂+2R—NH₂→R—NH₃⁺+R—NHCO₂⁻

The experimental results reported below indicate that the monochalcogenide formation reaction could benefit from the presence of appropriate amounts of the charged forms of the primary amine solvent, such as the corresponding ammonium chloride and the ammonium-carbamate pair surfactants, because specific concentrations of these charged surfactants stabilize π-crystal faces and increase the selectivity of the reaction towards the cubic phase. That is, by stabilization of the cubic π-phase over the orthorhombic α-phase: it appears that in designated concentrations, these ligands bind preferentially to the π-phase surfaces due to the presence of dangling bonds, resulting in reduction of the surface energy and thermodynamic stabilization of the π-phase. However, as shown by the experimental results below, excessive amount of the charged surfactant in the primary amine solvent may turn to be unfavorable, reverting to the predominance of the α-phase in the reaction product. The invention is therefore based on supplying to the reaction mixture effective amounts of “beneficial contaminants” hitherto a major source of irreproducibility in the synthesis of monochalcogenides, to achieve polymorphic phase and shape control.

Accordingly, the invention is primarily directed to a process for preparing tin or germanium monochalcogenides of cubic crystal structure, the process comprises:

combining a source of tin or germanium and a source of chalcogen in a reaction vessel in the presence of uncharged liquid primary amine R—NH₂ and a charged form R—NH₃⁺ associated with a counter anion, wherein R is saturated or unsaturated hydrocarbyl, which may be the same or different in the uncharged and charged forms, and recovering from the reaction mixture an essentially polymorphically pure cubic monochalcogenide.

The process is preferably directed to the preparation of cubic tin sulfide and cubic tin selenide. Suitable tin sources are stannous salts, which can dissociate in the organic reaction medium to supply Sn²⁺ ions, e.g., stannous halides, such as stannous chloride. Suitable chalcogen sources include compounds which are miscible with, and inert towards, the organic reaction medium, such as NH₂C(S)NH₂ and NH₂C(Se)NH₂ (thiourea and selenourea, respectively), thioacetamide, selenoacetamide, elemental Se and elemental S.

The reaction takes place using liquid (or molten) primary amine R—NH₂ as the reaction solvent, preferably under anhydrous conditions. That is, a saturated or unsaturated primary amine maintaining liquid form at the reaction temperature (including molten amine) can be used as the reaction solvent. R is most preferably an alkyl or alkenyl that contain not less than 8 carbon atoms, e.g., from 8 to 20 carbon atoms. Preferred R groups are straight unsaturated or polyunsaturated chains, e.g., straight C12-C18 alkenyl groups. We obtained good results using primary amine bearing the oleyl group, CH₃(CH₂)₇CH═CH(CH₂)₇CH₂—NH₂.

The reaction is conveniently carried out by dissolving a source of tin in the primary amine R—NH₂ (at concentration up to saturation, preferably from 30 to 40 mM), to form a first solution, and separately dissolving the chalcogen in the primary amine R—NH₂ (at a concentration up to saturation, preferably from 30 to 40 mM for thiourea and 15 mM to 30 mM for selenourea), to form a second solution.

Dissolution of stannous halide salts in the primary amine solvent to produce the first solution (Sn precursor) is performed under heating, at a temperature in the range from 160 to 200° C., for example, around 180° C. Dissolution of NH₂C(S)NH₂ in oleylamine to produce the second solution (S precursor) also requires heating, e.g., to about 170° C. NH₂C(Se)NH₂ readily dissolves in the primary amine solvent even at room temperature, with the aid of sonication, to give the second solution (Se precursor).

Next, the second solution consisting of the sulfide or selenide precursor is rapidly combined (for example, up to a few seconds) with the first solution consisting of the tin precursor, e.g., by injection to the first solution, held at the elevated reaction temperature, which is usually not less than 160° C. for tin sulfide formation (e.g., from 160 to 200° C.) and not less than 90° (e.g., from 90 to 130° C.) for tin selenide formation.

The protonated form R—NH₃⁺ is associated with a counter anion such as halide, or the corresponding ammonium carbamate R—NHCO₂⁻. The protonated form can be supplied to the monochalcogenide formation reaction in different ways. For example, by addition of ex-situ prepared quaternary ammonium salt of the formula R—NH₃⁺Hal⁻. The preparation of such salts is well known in the art. For example, the ammonium salt formation reaction may be accomplished under anhydrous conditions by bubbling gaseous hydrogen halide into the liquid free amine. Completion of the reaction is marked by gas evolution, indicating that all available amine groups underwent protonation. R—NH₃⁺Hal⁻ salts formation reaction may also take place in a solvent such as ethanol, followed by separating the salt, washing, and drying to collect the solid salt form that can be used as an additive to the reaction mixture of the present invention.

The use of ex-situ prepared R—NH₃⁺Hal⁻ offers several advantages on industrial scale, since metered amounts of the charged surfactants can be readily supplied to the reaction mixture to meet the reaction's demand for accurate concentrations of the charged species in the reaction mixture. For example, such ex-situ prepared R—NH₃⁺Hal⁻ salts can be conveniently added in an appropriate amount to the tin precursor solution before the injection of the chalcogen precursor solution thereinto. As pointed out above, R may be the same or different in the uncharged amine and charged ammonium forms. Hence, when R—NH₃⁺Hal⁻ is added ex-situ, the R groups may not be necessarily the same. For example, similar R—NH₂ and R—NH₃⁺Hal⁻ may differ in the chain length (±1-3 carbon atoms difference) or the degree of saturation/unsaturation (number of C═C bonds along the chain).

However, it is generally preferred that the R—NH₂ and R—NH₃ forms have the same hydrocarbyl group.

When the protonated form R—NH₃⁺ is associated with ammonium carbamate as its counter anion, this pair consisting of [R—NH₃⁺+R—NHCO₂⁻] can be prepared ex-situ, by saturating the free amine with gaseous CO₂ to reach chemical equilibrium. Such ex-situ prepared [R—NH₃⁺+R—NHCO₂⁻] pair is well suited for use in the present invention. However, owing to the reversibility of the reaction of primary amines with CO₂, it may be more convenient to supply the [R—NH₃⁺+R—NHCO₂⁻] pair to the reaction mixture in-situ.

That is, the charged ammonium R—NH³⁺ can be supplied to the reaction in-situ, by treating the primary amine R—NH₂ in the first solution and/or in the second solution with an acidic gas under anhydrous conditions. For example, by bubbling hydrogen halide to the first solution and/or the second solution, thereby converting the primary amine to the corresponding ammonium halide R—NH³⁺ Hal⁻ in-situ, or by bubbling or introducing carbon dioxide to the first solution and/or the second solution, thereby converting the primary amine to the corresponding pair of charged species of the formula [R—NH₃⁺+R—NHCO₂] in-situ.

The monochalcogenide formation reaction takes place in the presence of the charged species at temperature in the range from 160 to 200° C. for tin sulfide or 90 to 130° C. for tin selenide, reaching completion almost instantaneously. The reaction mixture is then worked-up to recover the product. To this end, the reaction mixture is quenched to room temperature by pouring the content of the reaction vessel into a suitable organic solvent (e.g., methanol, or a mixture of methanol and chloroform), from which the solid product is readily separable by conventional methods such as decantation and centrifugation. The washing/separation cycles may be repeated several times. Lastly, the washed solid is dried. The product is collected in the form of nanoparticles of sizes ranging from 10-300 nm for SnS and 10-100 nm for SnSe.

As pointed out above, a key feature of the invention is that the amounts of the primary amine solvent and the corresponding charged forms thereof are proportioned to maximize the polymorphic purity of the product in favor of the cubic phase. The polymorphic purity of the product can be determined using acceptable methods, such as X-ray powder diffraction. X-ray powder diffraction patterns of cubic tin sulfide and tin selenide are shown in FIG. 1. X-ray powder diffraction of cubic tin sulfide exhibits characteristics peaks at positions 23.0, 26.6, 35.4 2θ (degrees). X-ray powder diffraction of cubic tin selenide exhibits characteristics peaks at positions 22.3, 25.8, 29.8°, 30.7°, 31.6° and 34.3 2θ (degrees). X-ray powder diffraction of orthorhombic tin sulfide exhibits characteristics peaks at positions 22.0, 27.5, 31.5 2θ (degrees). X-ray powder diffraction of orthorhombic tin selenide exhibits characteristics peaks at positions 21.6, 25.4, 30.5 2θ (degrees); values measured using Cu Kα radiation.

The polymorphic purity of the product could also be measured using nuclear magnetic resonance spectroscopy, Raman spectroscopy at low temperature, optical absorption spectroscopy and x-ray absorption spectroscopy.

As pointed out above, the concentration of the R—NH₃⁺Hal⁻ or the [R—NH₃⁺+R—NHCO₂⁻] pair in the reaction mixture (consisting of the R—NH₂ solvent) is adjusted to maximize the polymorphic purity of the product in favor of the cubic polymorph.

Concerning the preparation of tin sulfide nanoparticles, essentially pure cubic tin sulfide is obtained in the presence of 0.1 to 0.7M R—NH₃⁺Hal⁻ in the R—NH₂ reaction solvent, for example, from 0.2 to 0.6M, e.g., when R is the oleyl group [or, expressed otherwise, in mixtures proportioned from 2:1 to 10:1 R—NH₂:R—NH₃⁺Hal⁻ ].

Concerning the preparation of tin selenide nanoparticles, it is noted that at lower concentrations of the chosen surfactant, either R—NH₃⁺Hal⁻ or the [R—NH₃⁺+R—NHCO₂] pair, the cubic phase nanoparticles appear with cube morphologies, while at higher concentrations of the added surfactant, the orthorhombic phase becomes dominant, with rod-like morphology. To produce essentially pure cubic tin sulfide, R—NH₃⁺Hal⁻ is preferably present at a concentration from 0.2 to 0.7M in the R—NH₂ reaction solvent, whereas [R—NH₃⁺+R—NHCO₂⁻] achieves the desired effect of good polymorphic purity at concentration from 0.1 to 0.2M.

Another approach to adding charge-bearing molecular forms to the reaction mixture consists of in-situ reacting the primary amine reaction solvent/surfactant (R—NH₂) with suitable amounts of carboxylic acid, for example, the corresponding carboxylic acid R—CCOH. A condensation reaction between the amine and carboxylic acid (e.g., oleylamine and oleic acid) occurs during the synthesis of the monochalcogenide, to yield the corresponding amide (oleylamide; OOA). It appears that the amide adsorbs preferentially onto the nanoparticle surface, stabilizing π-SnS over α-SnS by mechanism of modifying surface energies of the polymorphs.

The amide formation reaction between oleyl amine and the corresponding acid oleic acid is illustrated below (water molecule is released):

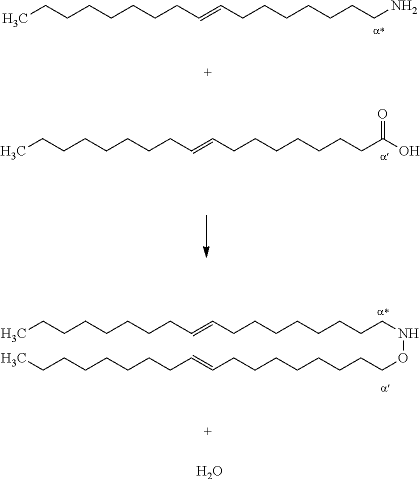 [SMILES: CCCCCCCC/C=C/CCCCCCCN ; CCCCCCCC/C=C/CCCCCCCC(=O)O ; CCCCCCCC/C=C/CCCCCCCCNOCCCCCCCC/C=C/CCCCCCCC ; O]

Amides are known to exist in resonance structures, one of which showing charge separation. The results of an NMR study conducted in support of this invention indicate that it the amide, i.e., the product of the reaction between oleyl amine and oleic acid, which adsorbs to the surface of the monochalcogenide nanoparticles.

Accordingly, another aspect of the invention is a process for preparing tin or germanium monochalcogenides of cubic crystalline structure, the process comprises combining a source of tin or germanium and a source of chalcogenide in a reaction vessel in the presence of uncharged liquid primary amine R—NH₂ and an additive which is R—COOH, wherein R is saturated or unsaturated hydrocarbyl, which may be the same or different in the uncharged and charged forms (as previously defined), and recovering from the reaction mixture an essentially pure cubic phase of the monochalcogenides. Instead of addition of the acid, the corresponding amide could be added (that is, an ex-situ prepared amide obtained from a reaction of amine R—NH₂ and R—COOH, namely, R—NH—C(O)—R).

The concentration of the added carboxylic acid is preferably not less than 0.1M, more preferably not less than 0.2 M, e.g., from 0.3 M to 1.0 M. Reaction conditions are as described above.

We acquired 1H and 13C NMR spectra of oleylamine and oleic acid in order to compare them with the NMR signal of cube shaped nanoparticles synthesized using mixtures of oleylamine and oleic acid. The π-SnS nano-cubes were dispersed in deuterated chloroform and analyzed using NMR. H NMR oleylamine and oleic acid spectra were compared with the acquired data for the nanoparticle. This comparison shows that the nanoparticle spectra contains features that are present in oleylamine and oleic acid, yet they are shifted from their original location as expected for bound species. Specifically, we could differentiate between the α-amine (2.7 ppm) and α-carboxyl (2.25) moieties denoted here as α* and α′, respectively. Quantification of those moieties enabled us to deduce the ratio between oleylamine and oleic acid present on the nanoparticles surfaces as 1:1. This suggests that oleylamine and oleic acid co-adsorb on the nanoparticle surface.

Our next step was to try and conduct HMBC and HMQC experiments in order to verify the chemical species. HMQC experiment validates the affiliation of the α-amine and α-carboxyl moieties. From HMBC experiments a correlation could be found between the α* moieties centered around 3.2 ppm and the carbonyl peak centered around 172 ppm. This indicates that the amine and carboxyl moieties are spatially close to each other.

1h-13C HMBC experiments are designed to probe J2 and J3 interactions, only protons that are distant up to 3 bonds away from the carbons are detected while signal from protons that reside directly on carbons is suppressed. For the above correlation to exist, the amine proton (α*) and carbonyl carbon must be closer than the 3 bonds relation which is not the case for oleylamine and OA pairs. This discrepancy could be settled if a condensation reaction between oleylamine and oleic acid is considered. The condensation of oleylamine and OA is expected to result in the formation of N-(cis-9-octadecenyl) oleamide (OOA) according to the reaction pathway depicted above.

The nanoparticles solution was centrifuged in order to precipitate out the nanoparticles from the solution. We continued by analyzing the remaining solvent using 1H-NMR. The results show that this solution contain the same moieties that were present in the solution containing the nanoparticles. In addition to low SNR which indicate on the low concentration of organic molecules, the only difference is found in the ratio between α* and α′. This points out that the chemical spices that are found in the solution are different from those present on the nanoparticles. Since the nanoparticles exhibit 1:1 ratio of α*:α′ we concluded the OOA is present on the nanoparticles surface.

### BRIEF DESCRIPTION OF THE DRAWINGS

FIG. 1 shows the XRPD pattern of π-SnS.

FIG. 2 shows the XRPD pattern of π-SnSe.

FIG. 3 presents 1D 1H NMR spectra of oleylamine, oleylammonium chloride and oleylammonium carbamate.

FIG. 4 provides TEM images (4a-4f) and corresponding XRPD patterns (4h-4j) of tin sulfide produced by Examples 1 to 3 and reference XRPD patterns (4g-4k) of pure phase tin sulfide (α-SnS and π-SnS), illustrating the effect of addition of ex-situ prepared oleylammonium chloride to the reaction mixture.

FIG. 5 provides TEM images (5a-5d) and corresponding XRPD patterns (5e-5h) of tin selenide produced by Examples 4 to 7, illustrating the effect of addition of ex-situ prepared oleylammonium chloride to the reaction mixture.

FIG. 6 is a TGA curve of oleylamine exposed to CO₂ at 110° C. The mass gain is expressed as percentage of mass gained with respect to the initial mass. The molar concentrations were calculated from this mass gain curve.

FIG. 7 provides TEM images (7a-7d) and corresponding XRPD patterns (7e-7h) of tin selenide produced by Examples 8 to 11, illustrating the effect of in-situ formation of oleylammonium-oleyl carbamate in the reaction mixture.

FIG. 8 provides TEM and SEM micrographs (8a-8g) and X-ray diffractograms (8h-8l) of tin sulfide produced by Examples 12 to 16, testing the effect of addition of oleic acid to the reaction mixture.

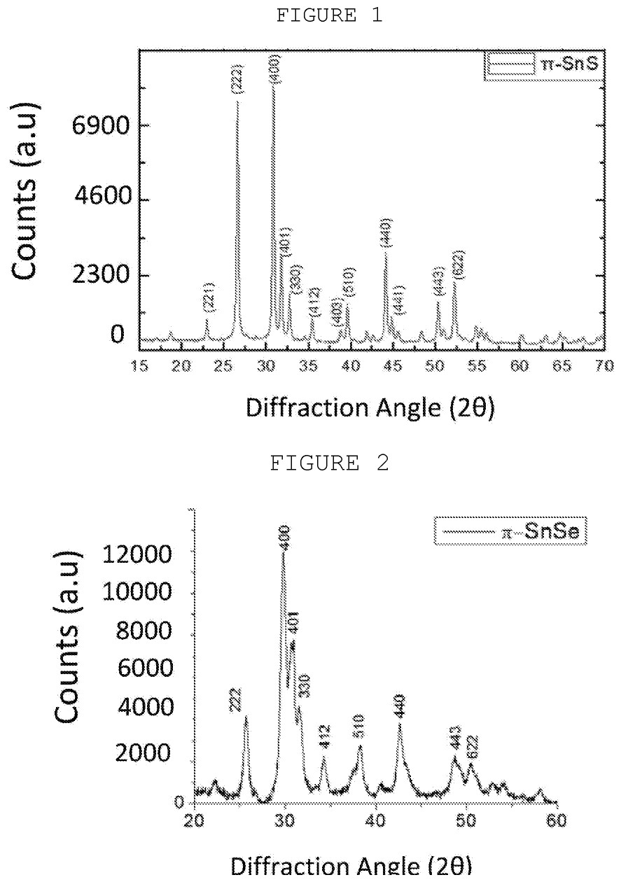

### EXAMPLES

Methods

Transmission electron microscopy (TEM) was carried out using a Tecnai G2 instrument operating at 120 kV. TEM samples were prepared by solvent evaporation from chloroform suspensions. Powder X-ray Diffraction (XRD) was performed on a Panalytical Empyrean powder diffractometer equipped with a position sensitive X'Celerator detector using Cu Kα radiation (λ=1.5418 Å).

Nuclear Magnetic Resonance (NMR) spectra were recorded on a Bruker AVANCE III 500 MHz spectrometer equipped with broad-band 1H decoupling probe (BBO) at room temperature. NMR samples were prepared by adding 0.25 g (for solid samples) or 0.3 mL (for liquid samples) to 10 mL of CDCl3 in a glass vial. The samples were completely dissolved in the solvent, and 0.5 mL of that solution was transferred to an NMR tube for analysis. Chemical shifts are given in parts per million relative to the residual solvent peak. Processing was carried out using Topspin 2.1 software.

High resolution scanning electron microscopy (SEM) was carried out using a Thermo-Fisher Verios 460L.

Materials

Tin(II) chloride (SnCl₂, reagent grade, 98%), oleylamine (OLA, >98%), oleic acid (Tech grade, 90%), thiourea (reagent grade, 98%), selenourea (reagent grade, 98%) were purchased from Sigma-Aldrich and used without further purification with the exception of oleylamine, which was heated to 150° C. and degassed under vacuum for at least 3 h, after which it was stored in a glovebox. This was done in order to release CO₂ captured by the amine headgroup and to eliminate low boiling point impurities such as water. Hydrochloric acid (32%), sulfuric acid (95%), methanol (99.8%) and chloroform (99.9%) were purchased from Bio-Lab and used without further purification.

### Preparation 1

### Synthesis of Oleylammonium Chloride (OACl)

OACl was prepared by titrating hydrochloric acid over sulfuric acid while the evolved HCl gas was dried and bubbled into oleylamine. This process was stopped when bubbles started evolving from the oleylamine, indicating complete reaction with HCl. Standard Schlenk line reaction set-up was used for the reaction.

### Preparation 2

### Synthesis of Oleylammonium Oleylcarbamate (OAOC)

OAOC was prepared by bubbling CO₂ gas into purified oleylamine inside a closed vessel. This process was terminated when CO₂ bubbles evolved from the oleylamine, indicating complete conversion to OAOC. Standard Schlenk line reaction set-up was used for the reaction.

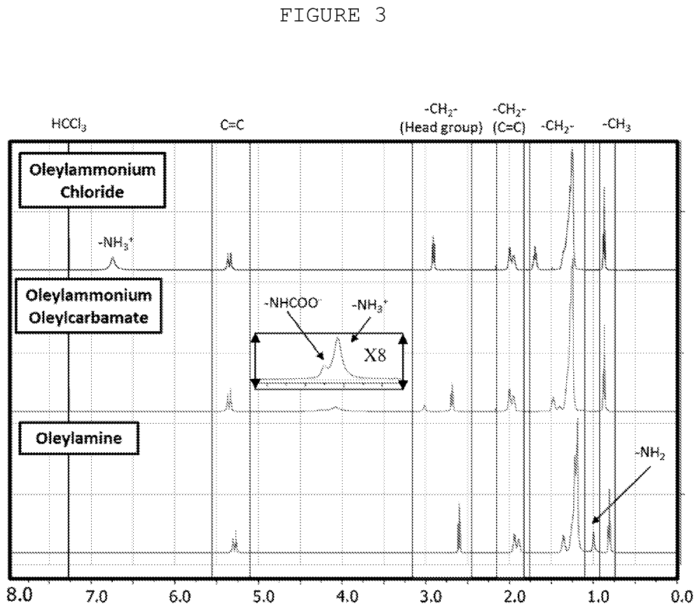

FIG. 3 presents 1D 1H NMR spectra of oleylamine, oleylammonium chloride and oleylammonium carbamate (the as-supplied oleylamine was purified according to the procedure described above).

The resultant NMR spectra obtained for oleylamine coincides with the chemical moieties expected from this molecule. The chemical shifts at 0.75, 1.95, and 5.23 ppm correspond to the methyl, allyl, and alkene groups, respectively. The range of chemical shifts from 1.1 to 1.5 ppm arises from protons that reside on the alkyl chain. The protons on the amine headgroup appear at 1 ppm and are marked with an arrow in FIG. 3.

Comparing the 1H NMR spectra of the purified oleylamine with that of oleylamine exposed to an excess of HCl gas shows complete transformation of oleylamine to oleylammonium chloride. The amine moiety is shifted downfield to 6.75 ppm and substantially broadened, indicating deshielding and chemical exchange with other equivalent protons. This is in accordance with the transformation of the amine group into ammonium. The α-amine protons are also shifted downfield as expected from reaction with the amine headgroup. Reaction of oleylamine with HCl can occur at the amine headgroup but is also possible at the C═C bond, resulting in possible formation of alkyl-chlorides. The ratio of the integrated signal from the methyl group and alkene group shows it remains unaffected after the reaction, ruling out reaction with the double bond and verifying reaction with the amine headgroup.

The 1H NMR spectrum of purified oleylamine reacted with an excess of CO₂ gas confirms complete conversion of the amine headgroups to oleylammonium-oleylcarbamate molecular pairs. The amine moiety is shifted to a broad pair of peaks around 4.2 ppm. These peaks correspond to protons that reside directly on the nitrogen in carbamate and ammonium head groups, as shown in the inset in the second row in FIG. 3.

### Examples 1 to 3

### Synthesis of SnS in Oleyl Amine and in a Medium Consisting of Oleyl Amine and Ex-Situ Prepared Oleyl Ammonium Chloride

General Procedure

56.8 mg of SnCl₂ and 5.5 ml of oleyl amine were placed in a 3-necked flask in a glove-box and transferred to the Schleck-line. 22.83 mg of thiourea were dissolved in 3 ml of oleyl amine and placed in a 1-necked flask in a glove-box and transferred to the Schleck-line. Glove-box was used in order to prevent moisture to react with the precursors. The Sn-precursor was heated to 180° C. for 1 hr, until the SnCl₂ completely dissolved. Meanwhile the S precursor was heated to 170° C. for 1 hr and injected to the Sn precursor. Instantaneous color change to deep-brown indicated the occurrence of the reaction.

The reaction was terminated by removing the reaction flask from the heating mantle and immediately quenching it to RT by pouring the content of the flask into a 50 mL test tube which was filled with methanol. The test tube was centrifuged at 2800 rpm for 5 min which after the solution was decanted. The test tube was filled with a mixture of 1:10 of chloroform and methanol and the process of washing and centrifuging was repeated two more times. The washed nanoparticles were then dried in a ventilated area and kept in powder form for storage.

Oleyl Amine/Oleyl Ammonium Chloride Reaction Medium

To test the effect of a combination consisting of a free primary amine and its chloride salt, the general procedure was repeated but oleyl ammonium chloride of Preparation 1 was added to the synthesis by weighing specific amounts of and subtracting the same number of moles of oleyl amine. Oleyl ammonium chloride was added to the Sn-precursor flask while handled in a glovebox.

The compositions of the reaction medium tested, consisting of free amine or mixtures of free amine/chloride salt are tabulated in Table 1. The compositions are expressed in terms of molar fractions of the free amine/chloride salt.

TABLE 1

| | Oleyl amine | Oleyl ammonium chloride |
|---|---|---|
| Example | (mole fraction) | (mole fraction) |
| 1 | 100 | 0 |
| 2 | 91 | 9 |
| 3 | 75 | 25 |

Results

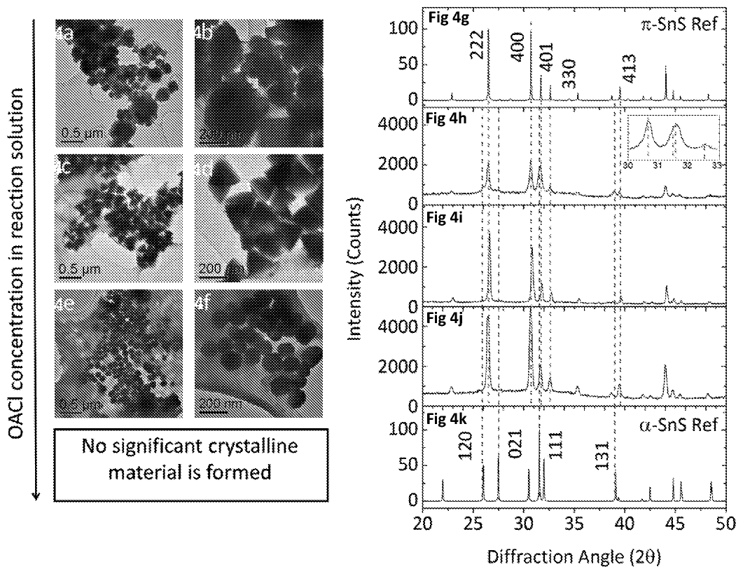

The results are shown by TEM micrographs (FIGS. 4a-4f) and corresponding XRD diffractograms (FIGS. 4g-4k).

Starting with the TEM images, it is seen that in the absence of the chloride salt, i.e., using neat oleyl amine (Example 1), the reaction product consists of nanoparticles exhibiting irregular shapes and sizes (295±216 nm; FIGS. 4a and 4b). Addition of low amount of the chloride salt, namely, carrying out the reaction in the presence of a mixture consisting of the free amine:chloride salt mixture at 10:1 molar ratio (Example 2), results in tetrahedron-shaped nanoparticles that are more uniform in size (183±23 nm; FIGS. 4c and 4d). Further increase the amount of chloride salt at the expense of the free amine, that is, when the reaction medium consists of 3:1 proportioned mixture (by moles, in favor of the free amine) induces shape transformation to rounded particles which are smaller in size and similar distribution (138±25 nm; FIGS. 4e and 4f).

Turning now to the XRD diffractograms, FIGS. 4g and 4k are simulated powder diffraction patterns of the pure π-SnS and α-SnS phases, whereas FIGS. 4h, 4i and 4j are the powder diffraction patterns of the experimentally obtained samples of Example 1, 2 and 3, respectively. The results indicate that particles synthesized using only oleyl amine consist of both cubic (n) and orthorhombic (a) phases (FIG. 4h). In contrast, particles synthesized using mixtures of oleyl amine and its chloride salt gave diffractograms consistent with that of the π-SnS crystal structure, as shown in FIGS. 4g-k. From this comparison we can conclude that the presence of oleyl ammonium chloride alongside the free amine stabilizes the π-phase of the nanoparticles and can result in powders consisting of pure π-SnS.

We have also tested for the extreme condition where neat oleyl ammonium chloride is used as the reaction medium. In this case injection of the precursor solutions did not result in the characteristic color change; the reaction solution remained transparent and no solid material could be centrifuged out from this solution. An aliquot was taken from the reaction mixture and transferred to a TEM grid for further analysis. TEM showed that the sample is composed mostly of large aggregates of amorphous material and some small crystalline particles, as confirmed by electron diffraction analysis (not shown).

### Examples 4 to 7

### Synthesis of SnSe in Oleyl Amine and in a Medium Consisting of Oleyl Amine and Ex-Situ Prepared Oleyl Ammonium Chloride

General Procedure

56.8 mg portion of SnCl₂, 5 mL of oleyl amine, and 0.5 mL of oleic acid were placed in a three necked flask in a glovebox and transferred to a Schlenk-line. An 18 mg portion of selenourea and 1.5 mL of oleyl amine were placed in an amber vial in a glovebox and transferred to a Schlenk-line. The flask and vial connected to the Schlenk line were cycled with inert gas and vacuum three times. After the solutions were subjected to vacuum, an inert atmosphere was flushed again to the flasks. The Sn precursor flask was heated to 180° C. using a heating mantle until the SnCl₂ completely dissolved and was kept at that temperature for 30 min. The temperature of the Sn precursor was lowered to 110° C. and kept for 30 min while the Se precursor was transferred to a benchtop sonicator and sonicated at room temperature for 30 min. The Se precursor was injected into the Sn precursor and the reaction was initiated, as indicated by instantaneous color change from transparent yellowish to opaque deep-brown solution.

The reaction was terminated by removing the reaction flask from the heating mantle and immediately quenching it to RT by pouring the content of the flask into a 50 mL test tube which was filled with methanol. The test tube was centrifuged at 2800 rpm for 5 min which after the solution was decanted. The test tube was filled with a mixture of 1:10 of chloroform and methanol and the process of washing and centrifuging was repeated two more times. The washed nanoparticles were then dried in a ventilated area and kept in powder form for storage.

Oleyl Amine/Oleyl Ammonium Chloride Reaction Medium

To test the effect of a combination consisting of a free primary amine and its chloride salt, the general procedure was repeated but oleyl ammonium chloride of Preparation 1 was added to the synthesis by weighing specific amounts thereof and subtracting the same number of moles of oleyl amine (from the solution of the Sn precursor). The compositions of the reaction medium tested, consisting of free amine or mixtures of free amine/chloride salt at various proportions, are tabulated in Table 1. The compositions are expressed in terms of molar concentration of the chloride salt in the free amine solvent (in the Sn solution).

TABLE 2

| Example | Oleyl ammonium chloride (M) |
|---|---|
| 4 | 0 |
| 5 | 0.4 |
| 6 | 0.75 |
| 7 | 1M |

Results

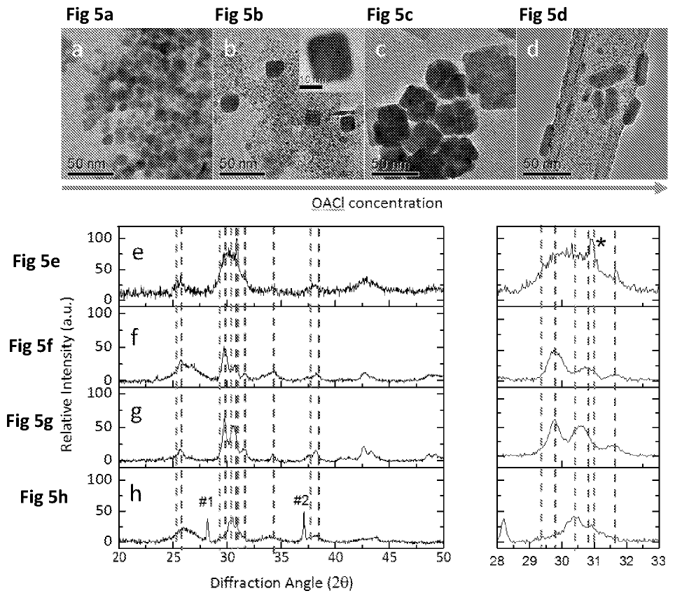

The results are shown by TEM micrographs (FIGS. 5a-5d), and corresponding XRD diffractograms (FIGS. 5e-5h), referring to Examples 4 to 7, respectively.

The TEM micrograph series shown in FIGS. 5a-d show that initially, the favored shape of the resultant nanoparticles is cubic, but rod-like nanoparticles dominate at the highest OACL concentration. For instance, the product of Example 5 (produced in the presence of 0.4M oleyl ammonium chloride) consists of nanoparticles the majority of which (75%) is cube shaped, with a minority of 11% platelets with additional 14% of other irregular morphologies. But on increasing the concentration of OACL to 1M (Example 7), rod-like particles with short edge of 13.5±1.8 nm and long edge of 40±4.2 become predominant: the 1M oleyl ammonium chloride concentration yielded majority of rods that consist about 85% of the synthesis product.

Turning now the corresponding XRPD patterns, it is seen that particles recovered from a reaction mixture devoid of oleyl ammonium chloride (Example 4) exhibit a broad peak around 2θ=30.27 degrees as seen in FIG. 5e. Increasing OACL content to 0.4M (Example 5) induces the emergence of new peaks at 20=angles of 29.8°, 30.7° and 31.6° as shown in FIG. 5f. These peaks that were obtained from a sample synthesized in the presence of 0.4M OACL match to (400), (401) and (330) Miller indices, three peaks which appear together as a signature mark for π-SnSe. Peak intensities agree well with the assignment to π-SnSe, as can be seen in the reference diffractogram in FIG. 5g, and the cube morphology of the nanoparticles correlates well with the cubic crystal system. As we further increase the OACL concentration to 0.75M (Example 6), we observe minor changes in the X-ray diffractogram presented in FIG. 5g. The intensity ratio of the (400), (401) and (330) reflection is changed due to increased intensity of the (401) reflection which is shifted to 2θ=30.5°, indicating gradual replacement of π-SnSe with α-SnSe since the strongest reflection of the former is centered around 2θ=30.2 degrees. The appearance of platelets in the TEM micrograph in FIG. 5c confirms this interpretation, as it is well established that α-SnSe nanoparticles usually appear in platelet morphology. Once the OACL concentration is further increased to 1M (Example 7), the dominant XRD peak reverts to the orthorhombic α-SnSe phase, as evident by the total disappearance of the π-SnSe peaks and the appearance of a new peak centered around 2θ=30.2° that corresponds to the (111) reflection of α-SnSe (FIG. 5h). Therefore, the rod-like nanoparticle morphology presented in FIG. 5d is clearly correlated with the orthorhombic α-SnSe phase.

The results indicated that with suitably proportioned mixture of R—NH₂/R—NH₃⁺Hal⁻ as the reaction medium, it is possible to achieve phase selective synthesis, which allows for stabilization of the cubic π-SnSe phase. The study reported in these Examples shows that a concentration of 0.2 to 0.6 M oleyl ammonium chloride in the primary amine reaction solvent affords nanoparticles population consisting essentially of pure crystalline phase cubic π-SnSe phase.

### Examples 8 to 11

### Synthesis of SnSe in Oleyl Amine and in a Medium Consisting of Oleyl Amine and In-Situ Formed Oleyl Ammonium-Oleyl Carbamate

General Procedure

The same general procedure of the previous set of Examples was used. However, in place of added oleyl ammonium chloride, the effect of in-situ formed oleyl ammonium-oleyl carbamate was tested. This pair of charged species was formed by exposing the solution of stannous chloride in oleyl amine to CO₂ for predetermined time periods. This exposure resulted in the following reaction:

CO₂+2CH₃(CH₂)₇CH═CH(CH₂)₇CH₂—NH₂→CH₃(CH₂)₇CH═CH(CH₂)₇CH₂—NH₃⁺+CH₃(CH₂)₇CH═CH(CH₂)₇CH₂—NHCO₂⁻

Subsequently, the Se solution was injected to the Sn solution.

Oleyl Amine/Oleyl Ammonium-Oleyl Carbamate Reaction Medium

The concentration of the oleyl ammonium-oleyl carbamate pair in the primary amine reaction solvent was varied by increasing the exposure time of the tin solution to CO₂, as tabulated in Table 3 (CO₂ was introduced into the Schlenk line]

TABLE 3

| | CO₂ Exposure | Oleyl ammonium - |
|---|---|---|
| Example | time (minutes) | oleyl carbamate (M) |
| 8 | 0 | 0 |
| 9 | 8 min | 0.15 |
| 10 | 12 min | 0.25 |
| 11 | 30 min | 0.63 |

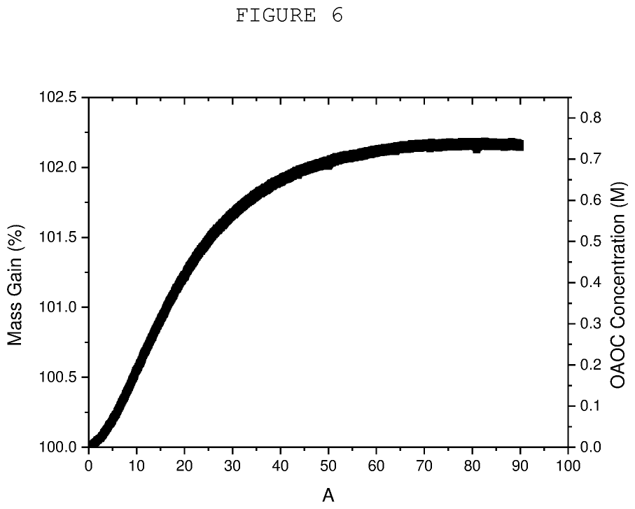

The molar concentrations of the oleyl ammonium-oleyl carbamate pair in the reaction mixture (in the Sn solution) set out in Table 3 were calculated from a calibration curve, shown in FIG. 6. The calibration curve was generated with the aid of thermogravimetric analysis (TGA). The TGA was performed with a TA Instruments Q500 analyzer, using an alumina crucible at a scan rate of 5° C./min. The oleylamine sample was placed in the alumina crucible that was constantly purged with argon gas. The sample was heated to 150° C. so that residual evolved CO₂ was effectively removed. The sample was then cooled to 110° C. and exposed to CO₂ at 110° C., and the mass gain was recorded over time. This temperature was chosen in order to simulate the absorption of CO₂ at the reaction temperature of colloidal synthesis of SnSe, which takes place at 110° C.—see general procedure (it is worthy of note that oleylamine can capture CO₂ until equilibrium is reached, depending on the equilibrium constant, which is temperature dependent).

The results were corrected to compensate for mass loss due to evaporation. The % mass gain of oleylamine owing to CO₂ intake was determined from the calibration curve of FIG. 6, and subsequently converted to molar concentration of OAOC tabulated in Table 3.

Results

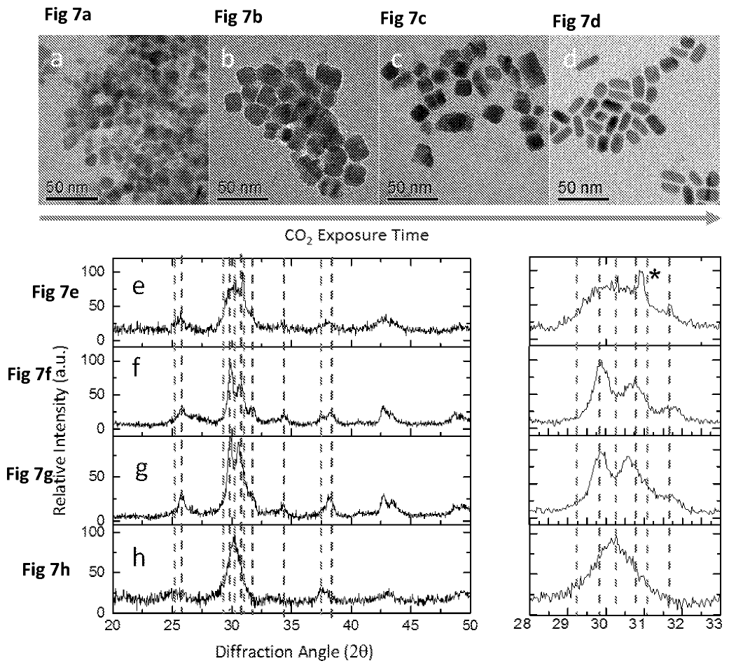

The results are shown by TEM micrographs of the SnSe nanoparticles (FIGS. 7a-7d), and corresponding XRD diffractograms (FIGS. 7e-7h), referring to the products of Examples 8 to 11, respectively. The magnified part of each diffractogram in the 29 range of 28-33° is shown respectively on the right. * denotes a SnSe2 impurity peak (JCPDS no. 23-0602). The red and blue lines indicate reference α-SnSe and π-SnSe peak positions, respectively.

Without exposure of the reaction mixture to CO₂ (Example 8), the nanoparticles formed appear to possess irregular shapes, large size distribution, and poor crystallinity. The corresponding TEM and XRD results are presented in FIGS. 7a and 7e, respectively.

Exposing the reaction mixture to CO₂ for 8 min (reaching concentration of 0.15 M OAOC—see Example 9) results in a cube-like nanoparticle morphology, as shown in the TEM micrograph presented in FIG. 7b. The size of the nanoparticles was 17±1.6 nm. The reaction product consists of 70% cube-like nanoparticles in admixture with other irregular morphologies. The corresponding X-ray diffractogram from this sample (FIG. 7f) matches to π-SnSe, as indicated by the emergence of the π-signature peaks at 29 angles of 29.8°, 30.7°, and 31.6° that correspond to π-SnSe (400), (401), and (330), respectively.

After exposure of the reaction mixture to CO₂ for 12 min, to reach 0.25 M OAOC—see Example 9), a subtle change is noticed in the XRD (FIG. 7g). The peak at 2θ=30.7° which corresponds to π-SnSe (401) shifts to 2θ=30.5°. This may be explained by appearance of α-SnSe as a minority phase in this sample. The strongest reflection of α-SnSe, (111), is at position 2θ=30.2°, and the presence of this phase would affect the XRD peak position by shifting the π-SnSe (401) peak to lower 20 angles.

Exposure of the reaction mixture to CO₂ for 37 minutes (resulting in buildup of 0.63 M OAOC), leads to significant changes. FIG. 7d is the TEM micrograph of the resultant SnSe nanoparticles, shows rod-like morphology of the nanoparticles, with a short edge size of 10±1.3 nm and long edge size of 20.7±2.7 nm. The rod-like nanoparticles constitute about 83% of the reaction product, while the rest show irregular morphologies. The corresponding XRD (FIG. 7h) shows that the intensity of the peak positioned around 30.2 increases at the expense of the π-SnSe peaks. This peak is attributed to the (111) reflection of α-SnS. A similar trend is noted by the peak positions at 20 of 37.4° and 38.3° that correspond to the (131) α-SnS and (413) π-SnSe Bragg reflections, respectively.

The results indicate that if the reaction takes place in a suitably proportioned mixture of R—NH₂/[R—NH₃⁺+R—NHCO₂⁻], stabilization of the cubic π-SnSe phase is achieved. The study reported in these Examples shows that a concentration of 0.1 to 0.2 M of oleyl ammonium-oleyl carbamate in the primary amine reaction solvent affords nanoparticles population consisting essentially of pure cubic π-SnSe phase.

### Examples 12 to 16

### Synthesis of SnS in Oleyl Amine and in a Medium Consisting of Oleyl Amine and Oleic Acid

General Procedure

56.8 mg of SnCl₂ and 5.5 ml of oleylamine were placed in a 3-necked flask in a glove-box and transferred to a Schlenk-line. 22 mg of thiourea and 3 ml of oleylamine were placed in 3-necked flask in a glove-box and transferred to a Schlenk-line. Connected flasks were cycled with inert gas and vacuum three times. The Sn and S precursors flasks was heated to 180° C. and 170° C. respectively using heating mantle. The precursors were kept at that temperature for 1 hr while SnCl₂ and thiourea were completely dissolved. The S precursor was injected into the Sn precursor and the reaction initiated, as indicated by instantaneous color change from transparent yellowish to opaque deep-brown solution.

The reaction was terminated by removing the reaction flask from the heating mantle and immediately quenching it to RT by pouring the content of the flask into a 50 ml test tube which was filled with methanol. The test tube was centrifuged at 2800 rpm for 5 minutes which after the solution was decanted. The test tube was filled with a mixture of 1:10 of chloroform and methanol and the process of washing and centrifuging was repeated two more times. The washed nanoparticles were then dried in a ventilated area and kept in powder form for storage.

Oleyl Amine/Oleic Acid Reaction Medium

To test the effect of a combination consisting of a free primary amine R—NH₂ and a corresponding carboxylic acid R—COOH, the general procedure was repeated but oleic acid (OA) was introduced to the reaction by systematically subtracting a well-defined amount of oleylamine volume and replacing it with the same amount of OA volume (the acid was added to the Sn-precursor flask while handled in a glovebox).

The compositions of the reaction medium tested, consisting of oleylamine or mixtures of the oleylamine and oleic acid are tabulated in Table 1. The compositions are expressed in terms of molar concentration of the chloride salt in the free amine solvent.

TABLE 4

| Example | Oleic acid (M) |
|---|---|
| 12 | 0 |
| 13 | 0.2 |
| 14 | 0.5 |
| 15 | 0.75 |
| 16 | 0.9 |

Results

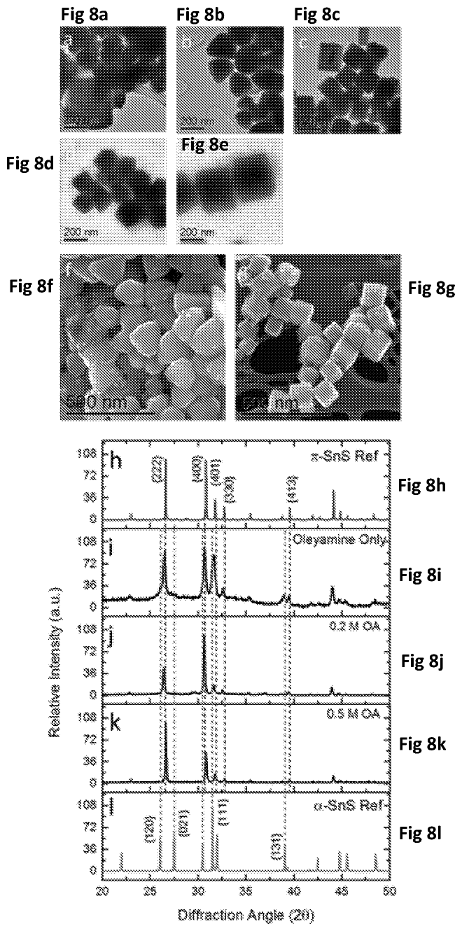

The results are shown by TEM micrographs of the SnS nanoparticles (FIGS. 8a-8e), SEM micrographs (FIGS. 8f-8g) and corresponding XRD diffractograms (FIGS. 8h-8l), referring to the products of Examples 8 to 11, respectively.

Without the addition of oleic acid (Example 12) the nanoparticles exhibit polydispersity in morphology and size (295±216 nm) as shown in FIG. 8a. The corresponding XRPD pattern (FIG. 8i) indicates that cubic and orthorhombic polymorphs coexist in the sample, suggested by the overlapping reflection from π-SnS 401 and α-SnS 111. The reflection overlap results in higher intensity at 2θ=31.6° compared to reference π-SnS XRD pattern. Additional support for the coexistence of both phases could be found by examining the α-SnS 120 and 131 reflections which appear at 2θ=26.06 and 2θ=38.9 respectively.

With the addition of oleic acid to the reaction mixture, the following trend was noted. Synthesis of SnS nanoparticles in the presence of 0.2 M OA (Example 13) induces the appearance of tetrahedral nanoparticles with better polydispersity. The morphology of the nanoparticles was determined by examining the TEM and SEM micrographs presented in FIGS. 8b and 8f, respectively. Increasing further the OA concentration to 0.5M (Example 14)→0.75M (Example 15)→0.9M (Example 16), resulted in the formation of SnS nano-cubes (FIGS. 8c, 8d, 8e, respectively; FIG. 8g). XRD of the samples that were synthesized in the presence of OA indicates the polymorphic purity of the product, which consist of π-SnS polymorph: the presence of the orthorhombic polymorphs was ruled out, as all of the peaks could be indexed as π-SnS.

The results indicated that with suitably proportioned mixture of R—NH₂/R—COOH as the reaction medium, it is possible to achieve phase selective synthesis, which allows for stabilization of the cubic π-SnS phase. The study reported in this Example shows that a concentration of 0.2 to 1.0 M of oleic acid in the primary amine reaction solvent (namely, the oleylamine) affords nanoparticles population consisting essentially of the π-SnS cubic polymorph.

1. A process for preparing tin or germanium monochalcogenides of cubic crystalline structure, the process comprises combining a source of tin or germanium and a source of chalcogenide in a reaction vessel in the presence of uncharged liquid primary amine R—NH₂ and a charged form R—NH₃⁺ associated with a counter anion, wherein R is saturated or unsaturated hydrocarbyl, which may be the same or different in the uncharged and charged forms, and recovering from the reaction mixture an essentially pure cubic phase of the monochalcogenides.

2. A process according to claim 1, wherein R is the same for the uncharged and charged forms.

3. A process according to claim 1, wherein the monochalcogenide is selected from the group consisting of tin sulfide and tin selenide.

4. A process according to claim 1, comprising dissolving a source of tin in the uncharged liquid primary amine R—NH₂, to form a first solution, dissolving a source of chalcogenide in the uncharged liquid primary amine R—NH₂, to form a second solution, and combining said first and second solutions, wherein the charged form R—NH₃⁺ is supplied to the first solution and/or the second solution.

5. A process according to claim 4, wherein the charged form R—NH₃⁺ is supplied to the reaction in the form of ex-situ prepared salt of the formula R—NH₃⁺ Hal−, wherein Hal is halide, added to the first solution and/or the second solution.

6. A process according to claim 4, wherein the charged form R—NH₃⁺ is supplied to the reaction in the form of ex-situ prepared pair of charged species of the formula [R—NH₃⁺+R—NHCO₂⁻], added to the first solution and/or the second solution.

7. A process according to claim 4, wherein the charged form R—NH₃⁺ is supplied to the reaction in-situ, by treating the uncharged liquid primary amine R—NH₂ in the first solution and/or in the second solution with an acidic gas under anhydrous conditions.

8. A process according to claim 7, comprising introducing hydrogen halide to the first solution and/or the second solution, thereby converting the primary amine to the corresponding ammonium halide R—NH₃⁺ Hal⁻ in-situ.

9. A process according to claim 7, comprising introducing carbon dioxide to the first solution and/or the second solution, thereby converting the primary amine to the corresponding pair of charged species of the formula [R—NH₃⁺+R—NHCO₂⁻] in-situ.

10. A process according to claim 3, wherein the source of tin is a stannous halide salt and the sources of sulfur and selenium are NH₂C(S)NH₂ and NH₂C(Se)NH₂, respectively.

11. A process according to claim 1, wherein the group R of the uncharged liquid primary amine R—NH₂ is alkyl or alkenyl that contains not less than 8 carbon atoms.

12. A process according to claim 11, wherein the R group is straight C12-C18 alkenyl group bearing one or more non-terminal carbon-carbon double bonds.

13. A process according to claim 1, wherein the uncharged liquid primary amine is oleyl amine and the charged form is oleyl ammonium associated with a counter anion selected from the group consisting of halides and oleyl carbamate.

14. A process according to claim 1, wherein the amounts of the uncharged primary amine and the corresponding charged form thereof are proportioned to maximize the polymorphic purity of the product in favor of the cubic phase.

15. A process for preparing tin or germanium monochalcogenides of cubic crystalline structure, the process comprises combining a source of tin or germanium and a source of chalcogenide in a reaction vessel in the presence of uncharged liquid primary amine R—NH₂ and an additive which is R—COOH, wherein R is saturated or unsaturated hydrocarbyl, which may be the same or different in the uncharged and charged forms, and recovering from the reaction mixture an essentially pure cubic phase of the monochalcogenides.
